(19) United States
(12) Patent Application Publication
(10) Pub. No.: US 2015/0064119 A1
(43) Pub. Date: Mar. 5, 2015

(54) COSMETIC ADDITIVES CONTAINING ALKALI PHOSPHATES

(71) Applicant: BK Giulini Chemie Gmbh, Ludwigshafen (DE)

(72) Inventors: Thauern Henrike, Weinheim (DE); Gabriele Brix, Ludwigshafen (DE); Thomas Staffel, Grunstadt (DE)

(73) Assignee: BK Giulini Chemie Gmbh, Ludwigshafen (DE)

(21) Appl. No.: 14/227,407

(22) Filed: Mar. 27, 2014

Related U.S. Application Data
(62) Division of application No. 13/405,900, filed on Feb. 27, 2012, now abandoned.

(30) Foreign Application Priority Data
Jan. 19, 2012 (DE) 20 2012 000 469.7

(51) Int. Cl. A61K 8/24; A61Q 17/04; A61Q 17/00; A61K 8/29

(52) U.S. Cl. CPC A61K 8/24; A61K 8/29; A61Q 17/04; A61Q 17/005; A61K 2800/74
USPC 424/59; 424/606; 423/312

(57) ABSTRACT

The invention relates to a cosmetic formulation comprising an additive agent. The additive agent having a water soluble linear alkali polyphosphate with a chain length of at least 4 to 50. The alkali polyphosphate exhibits complexing, dispersing, and antimicrobial effects. The alkali polyphosphate has a pH in the range of 6.5 to 8.5 (in aqueous solutions).

The present invention relates to cosmetic additives having a complexing, dispersing, and antimicrobial action.

Phosphates are well known in the state of the art, particularly in the food and detergents industry. The term “phosphates” is here understood to mean salts and/or esters of phosphoric acid, or the entire palette of phosphate salts, from ortho- and di-phosphates to the polyphosphates, and esters of these. In the context of the present invention, linear phosphates with at least 3 phosphate groups are of interest. Up to a chain length of 10, these are also known as oligophosphates. Polyphosphates can also be characterised with the P₂O₅ content. Table 1 gives an overview.

TABLE 1

| Chain length | Description | P₂O₅ content |
|---|---|---|
| 1 | Orthophosphate | 43% |
| 2 | Diphosphate | 53% |
| 3 | Triphosphate | 58% |
| 4-10 | Oligophosphate | 60-64% |
| >10 | Polyphosphate | 66-71% |

Based on the variety of their properties, the phosphates are utilised in very different applications. The best known applications, in addition to as fertilizers and as water treatment agents are in detergents and cleaning agents. Phosphates in detergents have been prohibited for more than 20 years in Germany, to prevent so-called eutrophisation of the waters which receive the waste. In certain specialised cleaning agents, such as e.g. dishwashing tablets, phosphates are still widely permitted and are in use. The function which makes them suitable for these applications is their complexing action on dissolved calcium and magnesium ions, resulting in reduction of the hardness of the water; and their soils dispersion action in the washing water.

In the state of the art, the use of phosphates in cosmetics has also been described.

According to DE 199 00 192 A1 phosphates (mentioned are diphosphates, triphosphates, and polyphosphates) may be added to salves, creams, make-ups, and lipsticks for preventive and curative purposes (with regard to fungal attack). DE 199 00 192 A1 does not contain demonstrations of effectiveness.

In DE 690 08 168 T2, the addition of phosphates to sun protection agents containing titanium dioxide as a UVA filter is described. The phosphates, added in an amount of 0.025 to 30 wt. %, associate spontaneously with the TiO₂ particles, i.e. a coating of phosphates is formed on the surface of the TiO₂ particles, which prevents discolouration by the TiO₂. DE 690 08 168 T2 suggests that all the phosphates are equally effective.

A problem in the formulation of cosmetic agents such as e.g. sun protection agents is the use of preservatives, such as parabens, often in large amounts, which can lead to allergic reactions in sensitive users. Also, large amounts of parabens may be toxicologically objectionable and therefore undesirable in cosmetic formulations.

It has now been found, surprisingly, [in connection with the invention,] that linear alkali phosphates in such a cosmetic formulation, with or without coating of the TiO₂ particles with phosphates, provide a significant antimicrobial effect, and additionally are very effective in dispersing and in the complexing of ions.

The present invention solves the above-described problems, by means of cosmetic additives having complexing, dispersing, and antimicrobial action, which additives are comprised of at least one liner alkali polyphosphate with a chain length of at least 3.

The invention proposes a mixture of at least one linear alkali polyphosphate with a chain length of at least 3 and a paraben as preservatives for cosmetics and sun protection agents containing the cosmetic additive and preservative. With this, one can substantially reduce the amount of paraben(s) used as preservatives.

The linear alkali polyphosphate preferably has a chain length of at least 4, particularly preferably at least 5. There is no upper limit to the chain length; in fact, alkali polyphosphates with chain lengths of 10, 15, or even up to 50 phosphate groups have been found to be particularly effective. Greater chain lengths do not display better effectiveness, however. Because the cost of synthesis increases with increasing chain length, alkali polyphosphates with chain lengths up to 50, particularly up to 15, are preferred. Here the term “chain length” refers to the mean chain length, which can be determined by, e.g., ³¹P solution NMR.

Preferably the described alkali polyphosphates comprise sodium- and/or potassium alkali polyphosphates. Particularly preferably, one may use alkali polyphosphates which are already approved as cosmetic ingredients, such as the substances available under the names Carephos N®, Carephos 322®, Carephos 244®, and Carephos 188® from the firm BK Giulini GmbH, of Germany.

Alkali phosphates can also be characterised by their P₂O₅ content and the pH of their aqueous solutions. According to the invention, the P₂O₅ content is in the range of 58-71%, preferably in the range of 60-70%, and particularly preferably in the range of 62-70%. Preferably the pH value of an aqueous solution of the inventively employed alkali polyphosphates is in the range 6.5-8.5, particularly preferably 7.0-7.5. The pH of alkali polyphosphates is determined by the ratio of alkali metal ions to the P₂O₅ content.

The inventive additives are preferably utilized in an amount of 0.005-10 wt. %, particularly preferably 0.001-2 wt. % [sic], based on the total weight of the formulation, as additives for dispersing, complexing, and preserving, of cosmetic and medicinal formulations.

The method of manufacturing the cosmetic and medicinal formulations is a known method. In addition to the inventively employed additives, ordinarily additional additives and components which are per se known may be employed. One might mention, e.g. colorants, pigments, cleaning agents, care agents, protective agents, fragrances, formulation aids, processing aids, etc. It is advantageous that the inventive additives can reduce the number of necessary ingredients, because the inventive additives perform multiple functions. Thus, other complexers, dispersants, and preservatives can be eliminated or their amounts can be substantially reduced.

Cosmetic formulations include O/W (oil in water) or W/O (water in oil) emulsions, containing or comprised of water and a lipid component, along with the inventive additives.

The lipid component is comprised of one or more fats and/or waxes. Suitable candidates are in principle all known lipids, particularly animal fats, vegetable fats and oils, hardened fats, synthetic triglycerides, solid and liquid waxes, and wax-like compounds, fatty alcohols, sterols, saturated and unsaturated hydrocarbons, and silicones. Particularly preferred are vegetable fats and oils, e.g. apricot kernel oil, argan oil, avocado oil, babassu oil, cottonseed oil, borage oil, candelilla wax, carnauba wax, cashew kernel oil, peanut oil, safflower oil, oat oil, hazelnut oil, jojoba oil, cocoa butter, coconut milk, coconut oil, pumpkin seed oil, butterfat, flax seed oil, macadamia nut oil, corn oil, almond oil, evening primrose oil, olive oil, palm kernel oil, palm oil, peach kernel oil, rapeseed oil, rice oil, castor oil, black locust seed oil, sesame oil, shea butter, soybean oil, sunflower oil, walnut oil, wheat germ oil, and animal fats such as butter and mink oil, as well as natural waxes such as beeswax and lanolin.

Typically, a cosmetic formulation with the inventive additive will additionally be comprised of one or more of the following ingredients:
anionic emulsifiers, e.g. sodium cetyl stearyl sulphate or glycerin fatty acid compounds esterified with hydroxyacids such as lactic acid or citric acid, or amino acids;
amphoteric emulsifiers, e.g. betaine and lecithin, and phospholipids and proteins, and their hydrolysates;
neutral emulsifiers, e.g. phosphoric acid alkyl esters, fatty acids, esters of polyhydric alcohols with free hydroxyl groups, polyglycerin esters and -ethers, ethoxylated mono- and diglycerides, macrogol fatty acid esters, partial fatty acid esters of sugars, sorbitan fatty acid esters, macrogol sorbitan fatty esters, macrogol sorbitan polysorbates, natural fat mixtures with high molecular weight alcohols, and silicone derivatives;
coemulsifiers, quasi-emulsifiers, and consistency agents, such as e.g. fatty alcohols, gum arabic, natural lipids (waxes and triglycerides), semi-synthetic lipids (waxes, triglycerides, and hardened fats), synthetic waxes or fats, free fatty acids and fatty alcohols, terpenes, sterols, saturated and unsaturated hydrocarbons, and silicones;
pigments and colorants, such as e.g. titanium dioxide, aluminium silicates, Pigment Red, Pigment Violet, Pigment Yellow, iron oxides and hydroxides, barium sulphate, bentonite, chromium oxide, calcium carbonate, copper phthalocyanine, ultramarine, iron oxide, zinc oxide, and manganese (III) ammonium diphosphate;
fragrances such as essential oils, and synthetic fragrances;
antioxidants;
complex-forming agents;
buffer substances and/or pH regulators;
substances such as e.g. UV filters, particularly titanium dioxide or zinc oxide, and organic filters which lead to undesired discoloration [sic] with metallic cations such as iron, particularly butyl methoxy dibenzoylmethane;
preservatives, particularly e.g. parabens, or benzoic acid, benzoic acid salts and esters, propanoic acid and salts, salicylic acid and salts, sorbic acid and salts, o-phenylphenol, sodium o-phenylphenylate, chlorobutanol, 3-acetyl-6-methyl-2,4(3H)-pyrandione and salts, 5-bromo-5-nitro-dioxane, 2-bromo-2-nitro-1,3-propanediol, triclosan, imidazolidinyl urea, poly(hexamethylenediguanide) hydrochloride, phenoxyethanol, quaternium 15, DMDM hydantoin, benzyl alcohol, piroctone olamine, 1,2-dibromo2,4-dicyanobutane, o-cymen-5-ol, methylchloro- or methylisothiazolinone, chloroacetamide [lit., “chlotacetamide”], chlorhexidine, cetrimonium chloride or bromide, diazolidinyl urea, chlorphenesin, and sodium hydroxymethylamino acetate.

The inventive additive is particularly suitable for formulations in which pigments are used, particularly formulations in which titanium dioxide is used, e.g. sun protection formulations. It is also suitable for formulations in which cations, such as of iron, can lead to undesired discoloration, particularly formulations which contain butylmethoxy dibenzoylmethane, e.g. sun protection formulations. It has been found to be particularly suitable in cosmetic formulations in which it is desirable to minimise the amount of preservatives such as parabens.

The cosmetic formulation may advantageously be of any desired consistency, from stable creams and salves to thinner flowable lotions and milks, to sprayable formulations.

The inventive additive can be used particularly advantageously in sun protection agents. These contain, in a suitable base, at least one light protection agent and the inventive additive. Typically they contain other additives such as preservatives, binders and/or opacifiers, viscosity regulators, etc., or in general combinations of these. The base may comprise known emulsions, creams, salves, gels, etc. The inventive additive may particularly advantageously be incorporated in the aqueous phase, particularly in emulsions. Emulsions, either oil-in-water or water-in-oil, are preferred bases.

Also, it has been found, surprisingly, that alkali polyphosphates work synergistically with parabens in their preservative action. Parabens are preservatives which are per se known, which are often used. However, for at least some persons, they are not well tolerated. According to the invention, by combining parabens with alkali polyphosphates, a smaller amount of parabens are used, without detriment to the preservative action. Common parabens which are suitable according to the invention are, e.g., methylparaben, propylparaben, benzylparaben, butylparaben [lit., “butalparaben”], ethylparaben, hexamidine paraben, isobutylparaben, and isodecylparaben; preferred are methyl and propylparaben.

The invention will be explained in more detail with reference to the following exemplary embodiments, which do not limit the scope of the invention. Unless stated otherwise or implied from the context, figures given in percent are percent by weight (wt. %), based on the weight of the mixture.

The invention also relates to all combinations of preferred variants, to the extent not mutually exclusive. The term “approximately” or “ca.” in a specification means that values at least 10% higher or lower are excluded, or 5% higher or lower, or in each case 1% higher or lower [sic].

The dispersing action was demonstrated in the following tests:

### EXAMPLE 1

First a model formulation was prepared, which contained an ingredient which formed agglomerates [sic]. This formulation is given in Table 2.

TABLE 2

| | Commercial | | |
|---|---|---|---|
| Phase | name | INCI Name | Amount (g) |
| A | Water, | Water | to make |
| | demineralised | | up 100 |
| | Glycerin | Glycerin | 3.00 |
| | Euxyl K 300 | Preservative [sic] | 0.50 |
| B | Keltrol CG-T | Xanthan gum | 0.30 |
| C | Cetiol CC | Dicaprylyl carbonate | 4.00 |
| | Cetiol Sensoft | Propylheptyl caprylate | 4.50 |
| | Cosmedia DC | Hydrogenated dimer | 1.00 |
| | | dilinelyl/ | |
| | | dimethylcarbonate | |
| | | copolymer | |
| | Neo Heliopan OS | Ethylhexyl salicylate | 7.50 |
| | Cosmedia Gel CC | Dicaprylyl carbonate | 2.00 |
| | | (and) stearalkonium | |
| | | hectorite (and) | |
| | | propylene carbonate | |
| D | Emulgin VL 75 | Lauryl glucoside (and) | 3.00 |
| | | polyglyceryl-2-di- | |
| | | polyhydroxy- | |
| | | stearate (and) glycerin | |
| | Emulgade PL | Cetaryl glucoside (and) | 2.50 |
| | 68/50 | cetaryl alcohol | |
| E | Titanium | Titanium dioxide | 15.00 |
| | dioxide E 171 | | |
| | EDTA | EDTA | 0 or 1.00 |
| | Sodium | Sodium polyphosphate | 0, 0.25, |
| | polyphosphate | | or 1.00 |

To produce the formulation, Phase B was dissolved in Phase A until a homogeneous mass was produced, which was then heated to a temperature of 75-80° C. Phase C was prepared, with Cosmedia Gel CC being dissolved in the rest of phase C, until a homogeneous phase resulted. This was also heated to ca. 75-80° C., and then Phase D was added, followed by Phase E with stirring.

The mixture of Phases. C, D, and E was added to the aqueous phase, and the mixture was stirred 5 min at 70 rpm. This mixture was homogenised at 60° C. with an Ultra Turrax stirrer at 11000 rpm, and then was stirred at 200 rpm until the temperature fell to room temperature.

After 24 hours, the dynamic viscosities of these formulations were determined at 25.4° C. with the aid of a “Thermo Haake Roto Visko 1” rotary viscometer at an approximate shear rate of 10 sec⁻¹; these figures are given in Table 3.

TABLE 3

| Emulsion | Phase E | Viscosity |
|---|---|---|
| 1 | 15 g Titanium dioxide E 171 | 1.74 Pa |
| | | sec |
| 2 | 15 g Titanium dioxide E 171 | 1.53 Pa |
| | 0.25 g Sodium polyphosphate | sec |
| 3 | 15 g Titanium dioxide E 171 | 1.34 Pa |
| | 1.00 g Sodium polyphosphate | sec |
| 4 | 15 g Titanium dioxide E 171 | 1.64 Pa |
| | 1.00 g EDTA | sec |

The formulations prepared in the described manner were stable according to the centrifuge test. The addition of sodium polyphosphates resulted in a clearly improved distribution of the TiO₂ particles, which were recognisable in a microscope and clearly visible at the lower viscosity.

The results illustrate that the addition of alkali polyphosphate in comparison to EDTA provided improved dispersion of the inorganic pigment in cosmetic suspensions.

### EXAMPLE 2

The antimicrobial action of the inventive polyphosphates was studied using a simple formulation described in Table 4.

TABLE 4

| | Commercial | | |
|---|---|---|---|
| Phase | name | INCI Name | Amount (g) |
| A | Cutina KD 16 | Glyceryl stearate SE | 12.00 |
| | Tegosoft HP | Isocetyl palmitate | 10.00 |
| B | Methylparaben | Methylparaben | 0 or 0.18 |
| | Propylparaben | Propylparaben | 0 or 0.05 |
| | Carephos N | Sodium | 0 or 1.00 |
| | | polyphosphate | |
| | Karion FP | Sorbitol | 25.00 |
| | Water | Water | to make |
| | | | up 100 |

Phases A and B were separately heated to 75° C. Phase B was slowly added to Phase A under stirring. This mixture was emulsified at 400-500 rpm. At 60° C., the mixture was homogenised with an Ultra Turrax stirrer at 6000 rpm, then cooled to room temperature. A total of 4 test emulsions were prepared:

Comparison Emulsion 1: Without parabens and without polyphosphate. pH 7.96.
Comparison Emulsion 2: With parabens but without polyphosphate. pH 8.22.
Emulsion 3: According to the invention, with polyphosphate but without parabens. pH 7.53.
Emulsion 4: According to the invention, with parabens and with polyphosphate. pH 7.89.

The determination of the antimicrobial effectiveness was carried out using the preservatives test according to the European Pharmacopoeia (Basic Part, 2008, 6th Edition, pub. Deutscher Apotheker Verlag, Stuttgart). According to the European Pharmacopoeia, the following test organisms should be used: Pseudomonas aeruginosa, Staphylococcus aureus, Candida albicans, and Aspergillus niger. Escherichia coli is mentioned as a reasonable substitute. A topical formulation is sufficiently well preserved if the criteria in Table 5, at least Criterion B, are fulfilled.

TABLE 5
Requirements for preparations for topical application:
Log reductions in the microorganism count

| | Criterion | 2 days | 7 days | 14 days | 28 days |
|---|---|---|---|---|---|
| Bacteria | A | 2 | 3 | — | No increase in |
| | | | | | bacterial count |
| | B | — | — | 3 | No increase in |
| | | | | | bacterial count |
| Fungi | A | — | — | 2 | No increase in |
| | | | | | fungal count |
| | B | — | — | 1 | No increase in |
| | | | | | fungal count |

In Tables 6-10, the colony numbers for the various tested emulsions are presented, based on weighted arithmetic averages.

TABLE 6
Bacterial count in CFU/g, for Escherichia coli:

| | Prior to | Days after addition | | | | | |
|---|---|---|---|---|---|---|---|
| Emulsion | addition | 0 | 2 | 7 | 14 | 21 | 28 |
| 1 | <10² | 7.5 10⁶ | 10⁶ | 9.5 10⁶ | 1.2 10⁶ | 9.5 10⁶ | 4.5 10⁶ |
| 2 | <10² | 4.3 10⁶ | 2.9 10⁵ | 10² | <10 | 10 | <10 |
| 3 | <10² | 1.4 10⁷ | 9 10⁶ | 1.2 10⁷ | 8.5 10⁶ | 1.4 10⁷ | 3.7 10⁶ |
| 4 | <10² | 2.2 10⁵ | 10² | <10 | <10 | <10 | <10 |

TABLE 7
Bacterial count in CFU/g, for Pseudomonas aeruginosa:

| | Prior to | Days after addition | | | | | |
|---|---|---|---|---|---|---|---|
| Emulsion | addition | 0 | 2 | 7 | 14 | 21 | 28 |
| 1 | <10² | 1 10⁷ | 1.6 10⁷ | 2.1 10⁷ | 1.6 10⁷ | 3 10⁷ | 1.8 10⁷ |
| 2 | <10² | 6 10⁶ | 2.6 10⁵ | 4.4 10⁵ | 2 10⁵ | 3 10⁵ | 9.5 10⁵ |
| 3 | <10² | 4.9 10⁶ | 1.6 10⁷ | 3 10⁷ | 3.3 10⁷ | 4.7 10⁷ | 2.4 10⁷ |
| 4 | <10² | 7 10⁵ | <10² | <10 | <10 | <10 | <10 |

TABLE 8
Bacterial count in CFU/g, for Staphylococcus aureus:

| | Prior to | Days after addition | | | | | |
|---|---|---|---|---|---|---|---|
| Emulsion | addition | 0 | 2 | 7 | 14 | 21 | 28 |
| 1 | <10² | 8 10⁶ | 7.5 10⁵ | 7.5 10⁵ | 2.5 10⁴ | 7.5 10² | 6.7 10¹ |
| 2 | <10² | 2.5 10⁶ | 7.1 10⁵ | 2 10⁴ | 5.5 10³ | <10² | 10² |
| 3 | <10² | 7.5 10⁶ | 1.2 10⁶ | 1 10⁵ | 2 10³ | <10² | 9.5 10¹ |
| 4 | <10² | 8.5 10⁶ | 4.6 10⁴ | 6 10² | <10 | <10 | <10 |

TABLE 9
Mould count in CFU/g, for Aspergillus niger:

| | Prior to | Days after addition | | | | | |
|---|---|---|---|---|---|---|---|
| Emulsion | addition | 0 | 2 | 7 | 14 | 21 | 28 |
| 1 | <10² | 1.7 10³ | 2 10³ | 2 10³ | 4.2 10³ | 3.9 10³ | 1.5 10⁴ |
| 2 | <10² | 9.5 10² | 9.5 10² | 6 10² | 10² | 7.1 10¹ | 1.9 10¹ |
| 3 | <10² | 1.7 10³ | 1.1 10³ | 1.5 10² | 1.1 10² | 4.5 10¹ | 7.1 10¹ |
| 4 | <10² | 1.4 10³ | 2.5 10² | 10² | <10 | <10 | <10 |

TABLE 10
Yeast count in CFU/g, for Candida albicans:

| | Prior to | Days after addition | | | | | |
|---|---|---|---|---|---|---|---|
| Emulsion | addition | 0 | 2 | 7 | 14 | 21 | 28 |
| 1 | <10² | 4.3 10⁴ | 2.4 10⁵ | 7.1 10⁵ | 3.8 10⁵ | 4.5 10⁵ | 5 10⁵ |
| 2 | <10² | 10⁵ | 1.2 10⁵ | 1.6 10⁵ | 6.2 10⁴ | 7.6 10⁴ | 6.6 10⁴ |
| 3 | <10² | 8.6 10⁴ | 3.7 10⁴ | 3.5 10² | 10² | <10² | <10 |
| 4 | <10² | 1.9 10⁵ | 1.6 10⁵ | 4.6 10³ | <10 | <10 | <10 |

It is seen that the effect of the combination of parabens with polyphosphate in Emulsion 4 [(for this Example 2)] showed effectiveness toward all of the microorganisms, and met Criterion A of the European Pharmacopoeia. For the fungi, the microbial count was reduced by at least 2 log reductions after 14 days, with an increased count thereafter. For the bacteria, the bacterial count was reduced by at least 2 log reductions after 2 days, and 3 log reductions after 7 days, with no increase thereafter. The recommended effectiveness according to the European Pharmacopoeia was provided by the combination of polyphosphate and parabens, with the polyphosphate having a positive effect on the preservation provided by the parabens.

With Emulsion 3, having polyphosphate but no parabens, there were reductions in the counts for Staphylococcus aureus, Aspergillus niger, and Candida albicans. Indeed, the bacterial count of Staphylococcus aureus was even reduced with Comparison Emulsion 1 without parabens and without polyphosphate. For Aspergillus niger and Candida albicans, with Emulsion 3 without parabens but with polyphosphate there was a reduction in the microbial count. In contrast to this, with Comparison Emulsion 1 without parabens or polyphosphate, no reduction in the microbial count was observed. Thus polyphosphate inhibits the growth and propagation of Aspergillus niger and Candida albicans.

With Comparison Emulsion 2, having parabens but no polyphosphate, the effectiveness criteria according to the European Pharmacopoeia were not satisfied. For the fungi, in order to at least satisfy Criterion B, the microbial count after 14 days must be reduced by one log reduction. This did not occur. For the bacteria, E. coli and Staphylococcus aureus, Criterion B was satisfied but not Criterion A Because no criteria were satisfied for Pseudomonas aeruginosa, the effectiveness against bacteria as well did not meet the criteria of the European Pharmacopoeia. An increased amount of parabens would have been needed.

### EXAMPLE 3

To demonstrate complex formation by phosphates, iron ions were added. These can enter formulations in practice by, e.g., pipes, mixtures, or raw materials. As a base formulation, the formulation of a sun protection agent with butyl methoxy dibenzoylmethane was used. This UVA filter forms an intensive red complex with traces of irons. The complexing effect was studied using the following formulation:

TABLE 11

| | Commercial | | |
|---|---|---|---|
| Phase | name | INCI name | Amount (g) |
| A | Demineralised | Water | to make |
| | water | | up 100 |
| | Glycerin | Glycerin | 3.00 |
| | Euxyl K 300 | Preservative [sic] | 0.50 |
| B | Keltrol CG-T | Xanthan Gum | 0.30 |
| | Veegum | Magnesium aluminium | 2.00 |
| | | silicate | |
| C | Cetiol CC | Dicaprylyl carbonate | 4.00 |
| | Cetiol Sensoft | Propylheptyl caprylate | 4.50 |
| | Cosmedia DC | Hydrogenated dimer | 1.00 |
| | | dilinelyl/ | |
| | | dimethylcarbonate | |
| | | copolymer | |
| | Neo Heliopan 303 | Octocrylene | 10.00 |
| | Neo Heliopan OS | Ethylhexyl salicylate | 7.50 |
| | Neo Heliopan 357 | Butyl methoxy | 5.00 |
| | | dibenzoylmethane | |
| | Cosmedia Gel CC | Dicaprylyl carbonate | 2.00 |
| | | (and) stearalkonium | |
| | | hectorite (and) | |
| | | propylene carbonate | |
| D | Emulgin VL 75 | Lauryl glucoside (and) | 3.00 |
| | | polyglyceryl-2-di- | |
| | | poly-hydroxy- | |
| | | stearate (and) glycerin | |
| | Emulgade PL | Cetaryl glucoside (and) | 2.50 |
| | 68/50 | cetaryl alcohol | |
| E | Eusolex T-AVO | Titanium dioxide, | 7.50 |
| | | silica | |

The aqueous phase of the formulation was prepared by dissolving Phase B in Phase A until a homogeneous phase resulted. This was brought to a temperature of ca. 75-80° C. Phase C was prepared by dissolving Cosmedia Gel CC in the rest of Phase C until a homogeneous phase resulted. This was heated to ca. 75-80° C., followed by addition of phase D, and then Phase E was stirred in. The mixture of phases C, D, and E was added to the aqueous phase, and this mixture was stirred 5 min at 750 rpm. Then the formulation was cooled to room temperature while stirring at 200 rpm, during which at ca. 60° C. the mixture was homogenised 1 min at 11000 rpm with the aid of an Ultra Turrax stirrer.

The following additives were added to the described formulation:

0.01 g iron (III) chloride hexahydrate;
0.01 g iron (III) chloride hexahydrate und 0.045 g Carephos N,
0.01 g iron (III) chloride hexahydrate und 0.09 g Carephos N,
0.01 g iron (III) chloride hexahydrate und 0.09 g Disodium EDTA;
0.01% iron (III) chloride hexahydrate und 0.045% Utanit AF;
0.01% iron (III) chloride hexahydrate und 0.09% Utanit AF;
0.01% iron (III) chloride hexahydrate und 0.09% Phoskadent Pyro.

The respective amount of iron (III) chloride hexahydrate, Carephos N (linear alkali polyphosphate), disodium EDTA, Utanit AF (acid diphosphate, for comparison), and [sic] Phoskadent Pyro (alkali diphosphate, for comparison) was worked into the aqueous phase and was removed from the water content [sic].

The evaluation was carried out by colour measurement. Using a Minolta Chroma-Meter CR 300, the colour status of the formulas was determined after 6 weeks. The measurements yielded values which allowed an objective comparison of the individual formulations. The measurements determined 3 values: L, a, and b. The L value describes lightness and darkness, with 0 representing perfect black and 100 perfect white. The a value describes the red-green value, and the b value describes the yellow-blue value. There are different prefix signs on the a and b values, in accordance with the present CIE Lab system, according to which no colour can be simultaneously reddish and greenish or simultaneously yellowish and bluish. Thus, −a represents green, +a represents red, −b represents blue, and +b represents yellow. In the measurements, the differences of the L, a, and b values are indicated with respect to the “white standard”, with the values L=98.19, a=−0.01, and b=+1.48 [sic]. The following results were obtained:

Formulation [without the described additives]:
L: −21.82; a: +1.07; b: +0.78.
   Formulation+0.01% Iron (III) chloride hexahydrate
L: −24.81; a: +5.18; b: +3.99
   Formulation+0.01% Iron (III) chloride hexahydrate+0.045% Carephos N
L: −24.58; a: +3.63; b: +2.82
   Formulation+0.01% Iron (III) chloride hexahydrate+0.09% Carephos N
L: −23.34; a: +3.37; b: +2.54
   Formulation+0.01% Iron (III) chloride hexahydrate+0.09% Disodium EDTA:
L: −26.44; a: +5.18; b: +4.10
   Formulation+0.01% Iron (III) chloride hexahydrate+0.045% Utanit AF
L: −23.46; a: +3.95; b: +3.17
   Formulation+0.01% Iron (III) chloride hexahydrate+0.09% Utanit AF
L: −23.67; a: +3.22; b: +2.30
   Formulation+0.01% Iron (III) chloride hexahydrate+0.09% Phoskadent Pyro
L: −25.41; a: +4.44; b: +3.28.

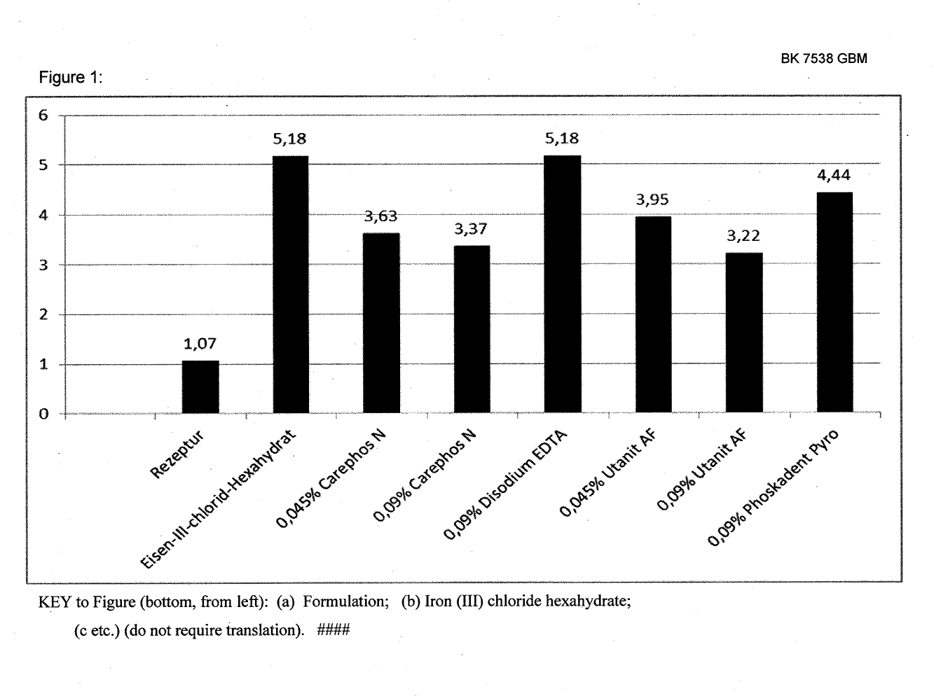

Because in this case red complexes are formed, particular attention should be paid to the a values, and in FIG. 1 they are shown graphically [(see last page)]. The higher the a value, the poorer is the complexing action. For comparison, the value for the formulation with 0.01% iron (III) chloride hexahydrate is presented. The best complexing performance was obtained with the acid phosphate Utanit AF. However, the formulations with Utanit AF were not sufficiently stable. The alkali phosphate Phoskadent Pyro gave satisfactory stability of the formulation, but its complexing effect was much reduced. The Phosphate Carephos N gave a complexing effect similar to that with the formulations with Utanit AF. The Carephos N formulations were also stable. Disodium EDTA in this example did not give any complexing effect.

It is surprising that alkali polyphosphates alone can provide good complexing of metal ions in cosmetic emulsions, along with good dispersion and preservation.

1-9. (canceled)

10. A cosmetic formulation comprising:
an additive agent comprising a water soluble linear alkali polyphosphate with a chain length of 4 to 50, wherein the alkali polyphosphate exhibits antimicrobial, complexing, and dispersing effects, and wherein the alkali polyphosphate has a pH in the range of 6.5 to 8.5 (in aqueous solutions).

11. The cosmetic formulation of claim 10, wherein the linear alkali polyphosphate has a chain length of 8 to 15.

12. The cosmetic formulation of claim 10, wherein the linear alkali polyphosphate is a sodium and/or potassium polyphosphate.

13. The cosmetic formulation of claim 10, wherein the alkali polyphosphate has about 58 to 71% polyphosphate.

14. The cosmetic formulation of claim 10, wherein the additive agent is in an amount of 0.005 to 10 wt. %, based on the total weight of the cosmetic formulation.

15. The cosmetic formulation of claim 14, wherein the additive agent is in an amount of 0.0012 wt. %, based on the total weight of the cosmetic formulation.

16. The cosmetic formulation of claim 10, further comprising a preservative.

17. The cosmetic formulation of claim 16, wherein the preservative is selected from a group comprising paraben, methylparaben, propylparaben, benzylparaben, butylparaben, butalparaben, ethylparaben, hexamidine paraben, isobutylparaben, and isodecylparaben.

18. The cosmetic formulation of claim 10, further comprising a light-protective substance.

19. The cosmetic formulation of claim 18, wherein the light-protective substance comprises titanium dioxide.

20. The cosmetic formulation of claim 10 is an emulsion, suspension, solution, cream, salve, gel, stick, or spray.

21. The cosmetic formulation of claim 10 having a viscosity of less than 1.64 Pa sec.

22. The cosmetic formulation of claim 10 having a pH of less than 7.96.

23. The cosmetic formulation of claim 10, wherein the additive agent inhibits the growth of bacteria, mould, and yeast.

24. The cosmetic formulation of claim 10, wherein the additive agent inhibits the growth of Escherichia coli, Pseudomonas aeruginosa, Staphylococcus aureus, Aspergillus niger, and Candida albicans.

25. The cosmetic formulation of claim 10 is an emulsion, suspension, solution, cream, salve, gel, stick, or spray.

26. The cosmetic formulation of claim 10, further comprises one or more of colorants, pigments, cleaning agents, care agent, protective agents, fragrances, formulation aids, and/or preparation aids.
